SEQUENCE LISTING (6 sequences)

SEQ ID NO: 1 - protein, 5 aa, Artificial sequence
  peptide sequence cleavable by proteinases present in tear fluid
LALLA

SEQ ID NO: 2 - protein, 8 aa, Artificial sequence
  peptide sequence cleavable by proteinases present in tear fluid
GPLALLAQ

SEQ ID NO: 3 - protein, 4 aa, Artificial sequence
  peptide sequence cleavable by proteinases present in tear fluid
ALAL

SEQ ID NO: 4 - protein, 8 aa, Artificial sequence
  peptide sequence cleavable by proteinases present in tear fluid
AAPRAARQ

SEQ ID NO: 5 - protein, 8 aa, Artificial sequence
  peptide sequence cleavable by proteinases present in tear fluid
AAPVAARQ

SEQ ID NO: 6 - protein, 6 aa, Artificial sequence
  peptide sequence cleavable by proteinases present in tear fluid
LLLAAG

(19) United States
(12) Patent Application Publication
(10) Pub. No.: US 2016/0175457 A1
(43) Pub. Date: Jun. 23, 2016

(54) OCULAR DEVICE

(71) Applicant: DSM IP ASSETS B.V., Heerlen (NL)

(72) Inventors: Luppo EDENS, Echt (NL); Peter Jan Leonard Mario QUAEDFLIEG, Echt (NL); James P. PARAKKA, Echt (NL); Petrus Johannes HERMSEN, Echt (NL); Dennis HEEMSKERK, Echt (NL); Michiel AKEROYD, Echt (NL)

(73) Assignee: DSM IP ASSETS B.V., Heerlen (NL)

(21) Appl. No.: 14/906,600

(22) PCT Filed: Sep. 12, 2014
(86) PCT No.: PCT/EP2014/069558

§ 371 Date: Jan. 21, 2016

Related U.S. Application Data
(60) Provisional application No. 61/877,135, filed on Sep. 12, 2013.
(60) Provisional application No. 61/978,390, filed on Apr. 11, 2014.

(51) Int. Cl. A61K 47/48; A61K 31/728; A61K 9/00

(52) U.S. Cl. CPC A61K 47/48338; A61K 9/0051; A61K 31/728

(57) ABSTRACT

The present invention relates to a composition, such as a coating composition, comprising a peptide linked to a lubricant, wherein the peptide is cleavable by one or more proteinases present in tear fluid. The invention further relates to an ocular device comprising said composition, such as a preformed contact lens.

### FIELD OF THE INVENTION

The present invention relates to a composition, in particular a coating composition, for use with an ocular device, such as a contact lens, and to an ocular device, such as a contact lens, which comprises the composition, e.g. onto which or into which the composition has been coated or incorporated. The invention also relates to methods for preparing the composition and ocular device, such as a contact lens, comprising the composition, e.g. onto which or into which the composition has been coated or incorporated.

### BACKGROUND TO THE INVENTION

Disposable contact lenses have become the most common type of contact lenses. They are worn for a specific period of time, then thrown out and replaced with fresh lenses. Many eye care practitioners and consumers prefer disposable contact lenses for their health and convenience benefits. The term “disposable” often refers to those contact lenses intended for daily replacement, those intended for replacement every one to two weeks and to those intended to be replaced monthly or quarterly.

A common source of confusion about contact lenses involves replacement and removal/wearing schedules. Replacement schedule refers to how often the contact lenses are discarded and replaced, whereas wearing schedule refers to how long the contact lenses may be worn before removing them. Non-compliance with recommended replacement and/or removal schedules may cause complications including deposits, mild wearing discomfort and vision-threatening adverse events.

Contact lens-related discomfort, especially late in the day or after prolonged wear, is a significant problem for many contact lens patients. Drop-out rates for contact lens wearers have been reported to be between 12 and 28% depending on the criteria used in different studies (Miller, W. L., Contact Lens Spectrum, July 2013). It is estimated that more than 50% of people who stop wearing contact lenses do so because of discomfort caused by dryness which is particularly high at the end of the day (Abelson, M. A., Review of Cornea and Contact Lenses, September 2012).

Accordingly, there is a need for new contact lenses that mitigate against the effects of non-compliance with replacement and removal schedules and that may also ameliorate end-of-day dryness.

### SUMMARY OF THE INVENTION

The present invention relates to a composition, in particular a coating composition, suitable for use with ocular devices, such as preformed contact lenses and ocular implants that come into contact with tear fluid. The composition comprises a lubricant, for example hyaluronic acid, linked to a peptide which is capable of being cleaved by one or more proteinases in tear fluid.

The composition may be coated on to or incorporated into an ocular device, such as a preformed contact lens: that is to say, a contact lens may comprise the composition of the invention (such as a coating composition). Typically, this means that the coating composition is covalently linked to the preformed contact lens. When the coating composition is coated onto or incorporated into a preformed contact lens and the contact lens is in use (i.e. applied to the ocular surface), one or more proteinases naturally present in tear fluid cleave the peptide linking the lubricant to the lens polymers so that the lubricant is liberated from the contact lens. Since eye irritations tend to increase proteinase levels in tear liquid, more lubricant will be released, thereby counteracting the irritation and relieving discomfort.

Lubricants that are added to or incorporated into lenses without a covalent attachment, tend to rapidly leach out into the tear fluid during wearing or into the sterile packaging solution during storage so that the time span of their lubricating activity is limited. But, in their free, non-immobilized form, the lubricants are better suited to moisturize, soothe and protect the ocular surface.

Accordingly, a contact lens of the invention provides a means for ameliorating the effects of, inter alia, non-compliance with replacement and/or removal schedules and ameliorating or overcoming end-of-day dryness.

According to the invention, there is thus provided a composition, such as a coating composition, comprising a peptide linked to a lubricant, for example hyaluronic acid, wherein the peptide is cleavable by one or more proteinases present in tear fluid.

The composition may be linked to a monomer, macromer or prepolymer suitable for use in the manufacture of a contact lens.

Where a composition of the invention comprises polymeric hyaluronic acid, due to the presence of the polymeric hyaluronic acid, the hyaluronic acid-peptide-monomer (eg. HEMA) conjugate is usually well soluble in water due to the multiple charges on the hyaluronic acid.

The invention also provides a composition comprising polymerizable vinylic group and a peptide linked to a lubricant (for example hyaluronic acid), that is covalently incorporated in to the contact lens, and wherein the peptide is cleavable by one or more proteinases present in tear fluid.

The invention also provides an ocular device, such as a contact lens or ocular implant which comes into contact with tear fluid, comprising a preformed contact lens or ocular implant that comes into contact with tear fluid and a composition according to the invention coated thereon or incorporated therein. That is to say, the composition is typically covalently linked to the ocular device.

The invention further provides:

a method for the manufacture of a composition, such as a coating composition, which method comprises linking a peptide to a lubricant, for example hyaluronic acid, wherein the peptide is cleavable by one or more proteinases present in tear fluid and, optionally, linking the resulting peptide-lubricant conjugate to a monomer, macromer or prepolymer suitable for use in the manufacture of a contact lens; and

a method for the manufacture of a composition, such as a coating composition which method comprises linking a peptide to a monomer, macromer or prepolymer suitable for use in the manufacture of a contact lens, wherein the peptide is cleavable by one or more proteinases present in tear fluid and linking the resulting peptide-monomer, -macromer or -prepolymer conjugate to a lubricant.

Also provided by the invention is a method for the manufacture of a contact lens which method comprises: providing a preformed contact lens; and coating said contact lens with a composition, such as a coating composition of the invention.

Where the composition, such as a coating composition, is linked to a monomer, macromer or prepolymer suitable for use in the manufacture of a contact lens, the invention provides a method for the manufacture of a contact lens which method comprises preparing a preformed contact lens in the presence of such a coating composition. The hyaluronic acid may be added before or after preparation of the preformed contact lens.

Accordingly, the invention provides a method for the manufacture of a contact lens which method comprises: preparing a composition, such as a coating composition, according to a method of the invention and preparing a preformed lens in the presence of the resulting composition, such as a coating composition.

Also, the invention provides a composition comprising a monomer, macromer or prepolymer suitable for use in the manufacture of an ocular device, such as a contact lens or ocular implant which comes into contact with tear fluid and a peptide cleavable by one or more proteinases present in tear fluid. The invention also provides an ocular device, such as a contact lens or ocular implant which comes into contact with tear fluid, comprising such a composition.

### BRIEF DESCRIPTION OF THE DRAWINGS

FIG. 1 shows peptide fragments formed by incubating linker peptide GPLALLAQ at 25° C. with A) tear fluid (over the weekend), B) human leukocyte elastase (30 minutes), C) human lung tryptase (overnight) and D) reference, peptide solution with no additions (over the weekend). The intact GPLALLAQ peptide with M+H+ 782.48 Da is shown on the right. Incubation of the peptide with tear fluid shows some degradation products (a.o. GPLAL/LAQ) that are similar to the degradation products formed upon incubation with elastase and tryptase.

FIG. 2 shows peptides AAPVAARQ and AAPRAARQ incubated with rinsing fluid of a single individual for 162 hours in the top panels. The bottom panels show the same peptides without rinsing fluid added. The data show that predominantly the N-terminal Ala is removed by incubation with rinsing fluid. For AAPVAARQ other low molecular weight peaks were observed, however only 544.32026 could be linked to one of the peptide fragments, being VAARQ. This observation implies that the only proteolytic cleavage initiated by tear fluid on peptide AAPVAARQ is hydrolysis of the peptide bond between Pro and Val. Cleavage C-terminal of Val does not occur. For AAPRAARQ both the singly charged and the doubly charged peptide is observed, both only showing cleavage at the N-terminal Ala. Hydrolysis of peptide bonds involving Arg (R) is not observed.

FIG. 3 shows the digestion pattern of peptide Leu-Leu-Leu-Ala-Ala-Gly (LLLAAG) incubated with human leukocyte elastase. The top panel shows the blank incubation without elastase, t=0 h is the sample analyzed directly after adding the elastase, t=0.5 h is the sample analyzed after thirty minutes of incubation and t=o.n. is the sample analyzed after incubation overnight. The data illustrate that the intact peptide LLLAAG, m/z 557.3625, is completely converted to LLLA, m/z 429.3045, upon an incubation with elastase.

FIG. 4 shows the peptide-HEMA fragments formed upon incubation of the conjugate with concentrated contact lens rinsing liquid of a single individual after 35 hours of incubation at 35° C. Panel A shows the absence of peptide-HEMA fragments in an extracted ion chromatogram of the control incubation without concentrated rinsing liquid. The mass accuracy was set to 10 ppm for the extracted ion chromatograms. Panel B shows the extracted ion chromatograms of the peptide-HEMA fragments for the incubation with concentrated rinsing liquid. Panel C shows the theoretical isotope pattern for the LAAG-HEMA fragment (top) and the isotope pattern measured (bottom). The identity of each of the peptide-HEMA fragments was confirmed by additional MS/MS experiments.

### DESCRIPTION OF THE SEQUENCE LISTING

SEQ ID NO: 1 sets out a peptide sequence which may be cleaved by proteinases present in tear fluid.

SEQ ID NO: 2 sets out a peptide sequence which may be cleaved by proteinases present in tear fluid.

SEQ ID NO: 3 sets out a peptide sequence which may be cleaved by proteinases present in tear fluid.

SEQ ID NO: 4 sets out a peptide sequence which may be cleaved by proteinases present in tear fluid.

SEQ ID NO: 5 sets out a peptide sequence which may be cleaved by proteinases present in tear fluid.

SEQ ID NO: 6 sets out a peptide sequence which may be cleaved by proteinases present in tear fluid.

### DETAILED DESCRIPTION OF THE INVENTION

Throughout the present specification and the accompanying claims, the words “comprise”, “include” and “having” and variations such as “comprises”, “comprising”, “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” may mean one element or more than one element.

The invention provides a composition, such as a coating composition, and a contact lens or ocular implant which comes into contact with tear fluid, which is coated with said composition. That is to say, a contact lens or ocular implant which comes into contact with tear fluid of the invention is one where the coating composition of the invention is disposed thereon, typically covalently bonded thereto.

A contact lens of the invention will typically be a disposable contact lens. Contact lenses generally fall into the following categories, based on how frequently they are replaced:
Disposable lenses: Replaced every two weeks, or sooner
Frequent replacement lenses: Replaced monthly or quarterly
Traditional (reusable) lenses: Replaced every six months or longer

Herein, the term “disposable” typically refers to both disposable and frequent replacement lenses.

Typically, a contact lens of the invention will comprise the coating of the invention coated onto a silicone hydrogel contact lens. The contact lens may be a preformed contact lens which is then coated with the coating composition of the invention. Alternatively, the coating composition may be coupled to a component used in the preparation of a contact lens. Use of such a component (to which the coating composition has been coupled) in the preparation of a contact lens results in a preformed contact lens on which the coating composition is disposed, on the surface thereof.

The coating composition is stable to lens processing/storage, but undergoes controlled degradation during use (i.e. wear) by one or more proteinases naturally present in tear fluid. The invention is partly based on the identification of protease activity in tear fluid which acts on the peptide present in the coating composition. As the protease activity in tear fluid is very low, another important aspect of the invention is the susceptibility of the peptide linker to the specific protease activity present. In other words, the amino acid sequence of the peptide linker is of paramount importance. The coating composition is slowly degraded during wear of the lens such that the lubricant is liberated.

By modifying the amino acid sequence of the peptide linker used, lubricant release can be adapted to specific needs. For example, disposable lenses will need higher lubricant release rates than frequently replaced lenses. Similarly the amino acid sequence of the peptide linker used may be adapted to the type of proteinase, for example a serine proteinase or a metallo proteinase, which is most prominent in the tear fluid of specific groups of individuals. Additionally the amino acid sequence of the peptide linker used may be adapted to promote or to slow down cleavage by a specific type of proteinase. See, for example, Kridel et al., J. Biol. Chem. 276(2) 20572-2-578, 2001, Rao et al., J. Biol. Chem. 266(15), 9540-9548(1991), Yasutake and Powers, Biochemistry 1981, 20, 3675-3679.

Metallo endopeptidases as well as serine endopeptidases can be active in tear fluids. In the human cornea, so called matrix metalloproteinases (MMP's) are secreted by epithelial cells, stromal cells and neutrophils. A.o MMP's 1, 2, 8, 9 and 13 have been detected in tear fluid (de Souza et al., Genome Biology 2006, 7:R72; Ollivier et al., Veterinary Ophthalmology (2007) 10, 4, 199-206; Balasubramanian et al., Clin Exp Optom 2013; 96:214-218; Zhou et al., Journal of Proteomics 75 (2012)3877-3885). Noteworthy are the elevated levels of MMP-9 recorded for individuals suffering from dry eyes (Acera et al., Ophthalmic Res 2008; 40(6):315-321). The various MMP's are known to preferably cleave peptide bonds involving hydrophobic amino acids such as Ala, Leu and Phe but their substrate specificity seems to be conferred at much broader positions so that cleavage by a particular MMP is hard to predict. A peptide in the composition of the invention may thus comprise one or more of Ala, Leu and Phe.

Additionally serine endoproteases have been detected in tear fluid. The latter group of endoproteases can be subdivided in trypsin-like and elastase-like activities. The trypsin-like endoprotease tryptase is released by mast cells (Butrus et al., Ophthalmology, 1990, Vol 97, No 12, pp 1678-1683). The elastase-like activity includes leukocyte elastase and myeloblastin (de Souza et al., Genome Biology 2006, 7:R72).

Accordingly, in a composition according to the invention, the peptide linker may be cleavable by a serine proteinase or a metalloproteinase present in tear fluid

Hyaluronic acid is found naturally in the vitreous humor, synovial fluid, and many other locations in the body and functions primarily as a lubricant and volumizer. In recent years, considerable enthusiasm has developed for this natural polymer's ability to lubricate, moisturize, and protect the ocular surface.

Studies have demonstrated that long-term use of hyaluronic acid relieves dry eye symptoms and reduces ocular surface damage without triggering allergic reactions. This molecule is known to both bind water and weakly adsorb to the eye's epithelial layer (so that it is retained on the ocular surface). In theory, then, its benefits derive from its ability to remain on the ocular surface and to hold moisture there.

Accordingly, a lubricant, such as hyaluronic acid, may ameliorate discomfort caused by eye dryness experienced by wearers of contact lenses, which is particularly high at the end of the day. Also, hyaluronic acid may mitigate the effects of non-compliance with lenses contributing to discomfort among contact lens wearers caused by dryness, which is particularly high at the end of the day.

The invention thus provides an ocular device such as a contact lens or ocular implant that comes into contact with tear fluid, in particular a disposable silicone hydrogel contact lens, which comprises an ocular device, such as a preformed contact lens composed of a silicone hydrogel material, onto which is coated a composition, such as a coating composition, comprising a peptide linked to a lubricant, such as hyaluronic acid, wherein the peptide is cleavable by one or more proteinases present in tear fluid. That is to say, the composition of the invention may be covalently linked to an ocular device, such as a contact lens or ocular implant that comes into contact with tear fluid.

The coating composition of the invention comprises a lubricant. A lubricant is any substance which has a demulcent effect.

A demulcent is any substance capable of soothing inflamed or otherwise irritated areas of the ocular surface (i.e. epithelium). Typically, a lubricant will have a demulcent effect by targeting and protecting mucus membranes with its oily or mucilaginous consistency, typically by forming a film over the membrane.

A demulcent can enhance ocular surface lubricity, easing wear and tear on the ocular surface caused by an eyelid during the blink process. Also, the often mucilaginous makeup of demulcents provides them with a water-binding capacity that can help keep the ocular epithelium hydrated

The coating composition of the invention comprises a lubricant linked to a peptide.

Non-limiting examples of suitable lubricants are:

hyaluronic acid;

cellulose derivatives, for example such as carboxymethylcellulose, hydroxyethylcellulose, methylcellulose and hypromellose;

dextrans;

polymeric alcohols, for example glycerin, polyethylene glycols (PEG), polysorbates and propylene glycol;

polyvinyl alcohols; and

povidone (polyvinylpyrrolidone).

A carbohydrate based lubricant can be linked to the peptide—either to the C-terminal carboxylic acid function of the peptide or the N-terminal amino function of the peptide or to a side chain functionality of one of its amino acid residues, optionally via a suitable linker, for example according to the methodology described for hyaluronic acid in Example 3.

In the case of alcoholic lubricants such as PEG, activation of the hydroxyl moiety is required in order to allow linking to an amine function of the peptide. Non-limiting examples of activation methods are treatment with triphosgene or bis-(2,5-dioxopyrrolidin-1-yl) carbonate. Attachment of the alcoholic lubricant to a carboxylic acid moiety of the peptide can be achieved via esterification. Non-limiting examples of achieving this are activation of a suitably N-protected peptide using pivaloyl chloride or isobutyl chloroformate and subsequent reaction with the hydroxyl function of the lubricant. Alternatively the coupling between the lubricant and a suitably N-protected peptide can be achieved using standards peptide coupling reagents, e.g. DCC, EDCl, T3P.

The molecular weight of the lubricant is dictated by two factors. First of all the molecular weight should be sufficiently high to ensure the required lubricating activity. On the other hand the molecular weight should be low enough to enable the synthesis and characterization of the lubricant-peptide conjugate (i.e. coating composition of the invention).

A molecular range of from about 200 Da to about 2 MDa can be used, more preferable the molecular range is between 1 and 20 kDa, most preferably the range is between 5 and 15 kDa.

For example, Dextran may have a molecular weight of about 70 kDa, carboxymethylcellulose may have a molecular weight of from 250 kDa to about 700 kDa, hydroxypropylmethylcellulose may have a molecular weight of form about 80 kDa to about 100 kDa, PEG may have a molecular weight of from about 300 Da to about 400 Da, polysorbates may have a molecular weight of about 1310 Da, Polyvinylalcohols may have a molecular weight of about 50 kDa. Povidone may have a molecular weight of from about 1000 kDa to about 1500 kDa.

Hyaluronic acid (also called hyaluronan or hyaluronate or HA) is an anionic, nonsulfated glycosaminoglycan distributed widely throughout connective, epithelial, and neural tissues. It is unique among glycosaminoglycans in that it is nonsulfated, forms in the plasma membrane instead of the Golgi, and can be very large, with its molecular weight often reaching the millions.

In a coating composition of the invention, the hyaluronic acid may have a molecular weight as set out above, for example of from about 1 kDa to about 1000 kDa, for example about 10 kDa.

The hyaluronic-peptide coating composition of the present invention may be characterized by the fact that the hyaluronic acid is covalently bound to the peptide, either to the C-terminal carboxylic acid function of the peptide or the N-terminal amino function of the peptide or to a side chain functionality of one of its amino acid residues, optionally via a suitable linker. Examples of side chain functionalities of amino acid residues are the ε-amino function of Lysine or the hydroxyl function of Serine.

Non-limiting examples of suitable linkers are linear and branched aliphatic C₂-C₂₄ diamines, amino alcohols and amino thiols; C₃-C₂₄ cycloaliphatic diamines, amino alcohols and amino thiols or C₆-C₂₄ aromatic and alkyl aromatic diamines, amino alcohols and amino thiols.

The hyaluronic-peptide coating compositions of the invention are accessible through coupling of the hyaluronic acid and the peptide in the presence or absence of a coupling agent to form a covalent bond or linkage under reaction conditions well known to a person skilled in the art. Non-limiting examples of coupling reactions are reductive amination of the terminal reducing carbohydrate—either directly (WO2004/004744) or after activation of the terminal residue by the reduction/limited oxidation method (U.S. Pat. No. 4,356,170) and amidation of the terminal reducing carbohydrate activated via the lactonization method (EPO454898) or esterification.

As used herein, the term “peptide” refers to a molecule comprising amino acid residues linked by peptide bonds and containing more than 2 amino acid residues, for example four, five, six, seven, eight, nine, ten or more amino acid residues. To prevent the development of any allergic reactions, the peptide length should typically be less than 10 amino acids, preferably less than 8 amino acids.

The amino acids are identified by either the single-letter or three-letter designations. The terms “protein” and “polypeptide” as used herein are synonymous with the term “peptide”. Thus, the terms “peptide”, “protein” and “polypeptide” can be used interchangeably. A peptide used in a composition of the invention may optionally be modified (e.g., glycosylated, phosphorylated, acylated, farnesylated, prenylated, sulfonated, and the like) to add functionality. Typically, the peptide will not exhibit enzymatic activity.

A composition according to any one of the preceding claims wherein the peptide comprises any one of the amino acid residues Ala, Ile, Leu, Phe, Asn, Gln, Pro, Gly or Val. The peptide may comprise one or more of such residues only.

In a preferred composition, the peptide comprises any one of the amino acid residues of Ala, Leu, Gln, Pro and Gly. The peptide may comprise one or more of such residues only.

Preferably, a Gly or Pro residue may be used to link the peptide to a monomer, macromere or prepolymer suitable for the manufacture of an ocular device, such as a contact lens.

Preferably, the peptide may not include a charged basic residue, such as Arg or Lys. The peptide may also not include a serine residue.

The sequence of the peptide may be any sequence which is capable of being cleaved by at least one proteinase present in tear fluid. Accordingly, in a coating composition of the invention, the peptide may be cleavable by one or more of endoproteases that have optimal activity at near neutral pH values. The internationally recognized schemes for the classification and nomenclature of enzymes from IUBMB include proteases. The updated IUBMB text for protease EC numbers can be found at the internet site: http://www.chem.qmw/ac.uk/iubmb/enzyme/EC3/4/11/.

The system categorises the proteases into endo- and exoproteases. An endoprotease is defined herein as an enzyme that hydrolyses peptide bonds in a polypeptide in an endo-fashion and belongs to the group EC 3.4. The endoproteases are divided into sub-subclasses on the basis of catalytic mechanism. There are sub-subclasses of serine endoproteases (EC 3.4.21), cysteine endoproteases (EC 3.4.22), aspartic endoproteases (EC 3.4.23), metalloendoproteases (EC 3.4.24) and threonine endoproteases (EC 3.4.25). Exoproteases are defined herein as enzymes that hydrolyze peptide bonds adjacent to a terminal α-amino group (“aminopeptidases”), or a peptide bond between the terminal carboxyl group and the penultimate amino acid (“carboxypeptidases”).

According to the data presented in Example 1 of the present application, the proteinases active in the tear fluid are either metallo endopeptidases (IUBMB enzyme class EC3.4.24) or serine endopeptidases (IUBMB enzyme class EC3.4.21). A number of scientific publications propose the presence of matrix metalloproteinases and stromelysins in tear fluid. Frequently mentioned serine proteinases include leukocyte and neutrophil elastase as well as myeloblastin and tryptase.

The peptide in a composition of the invention may be cleavable any one of these enzymes.

By modifying the amino acid sequence of the peptide linker used, lubricant release can be adapted to specific needs. For example, disposable lenses will need higher lubricant release rates than frequently replaced lenses. Similarly the amino acid sequence of the peptide linker used may be adapted to the type of proteinase, for example a serine proteinase or a metallo proteinase, which is most prominent in the tear fluid of specific groups of individuals. Additionally the amino acid sequence of the peptide linker used may be adapted to promote or to slow down cleavage by a specific type of proteinase. See, for example, Kridel et al., J. Biol. Chem. 276(2) 20572-2-578, 2001, Rao et al., J. Biol. Chem. 266(15), 9540-9548(1991), Yasutake and Powers, Biochemistry 1981, 20, 3675-3679.

A composition, such as a coating composition, of the invention may comprise the amino acid sequence as set out in any one of SEQ ID NOs: 1 to 6, but is not limited to any of those sequences.

The coating composition of the invention may be used to coat, or to impregnate,

a contact lens.

Accordingly, the invention provides a contact lens comprising:

a contact lens, typically a preformed contact lens; and

a coating composition of the invention coated onto or into the said preformed contact lens.

A preformed contact lens suitable for use in the invention may be any non-silicone or preferably silicone hydrogel contact lens. The coating composition may be coated onto an existing preformed contact lens. Alternatively, the coating composition may be coupled to a component used in the manufacture of a preformed contact lens such that manufacture of a preformed contact lens results in a preformed contact lens on which the coating composition is disposed (typically at least partially on the surface thereof).

Suitable preformed contact lenses are commercially available. Alternatively, a preformed contact lens (preferably a silicone hydrogel contact lens) can be made according to any methods well known to a person skilled in the art. For example, preformed contact lenses can be produced in a conventional “spin-casting mold,” as described for example in U.S. Pat. No. 3,408,429, or by the full cast-molding process in a static form, as described in U.S. Pat. Nos. 4,347,198; 5,508,317; 5,583,463; 5,789,464; and 5,849,810, or by lathe cutting of silicone hydrogel buttons as used in making customized contact lenses. In cast-molding, a lens formulation typically is dispensed into molds and cured (i.e., polymerized and/or crosslinked) in molds for making contact lenses. For production of preformed silicone hydrogel (SiHy) contact lenses, a SiHy lens formulation for cast-molding or spin-cast molding or for making SiHy rods used in lathe-cutting of contact lenses generally comprises at least one component selected from the group consisting of a silicone-containing vinylic monomer, a silicone-containing vinylic macromer, a silicone-containing prepolymer, a hydrophilic vinylic monomer, a hydrophobic vinylic monomer, a crosslinking agent (a compound having a molecular weight of about 700 Daltons or less and containing at least two ethylenically unsaturated groups), a free-radical initiator (photoinitiator or thermal initiator), a hydrophilic vinylic macromer/prepolymer, and combination thereof, as well known to a person skilled in the art.

The hyaluronic acid peptide conjugate (i.e. coating composition of the invention) may be coupled to one or more of the monomers/macromers/prepolymers described herein. A contact lens manufacture using such a coating composition-monomers/macromers/prepolymers will thus comprise a preformed contact lens and a coating composition (the latter disposed on or within the preformed contact lens).

Accordingly, the invention comprises a coating composition of the invention coupled to a monomer, macromer or prepolymer suitable for use in the manufacture of a contact lens.

A silicone hydrogel (SiHy) contact lens formulation can also comprise other necessary components known to a person skilled in the art, such as, for example, a UV-absorbing agent, a visibility tinting agent (e.g., dyes, pigments, or mixtures thereof), antimicrobial agents (e.g., preferably silver nanoparticles), a bioactive agent, leachable lubricants, leachable tear-stabilizing agents, and mixtures thereof, as known to a person skilled in the art. Resultant preformed SiHy contact lenses then can be subjected to extraction with an extraction solvent to remove unpolymerized components from the resultant lenses and to hydration process, as known by a person skilled in the art. In addition, a preformed SiHy contact lens can be a colored contact lens (i.e., a SiHy contact lens having at least one colored patterns printed thereon as well known to a person skilled in the art).

Many SiHy lens formulations are known and have been described in numerous patents and patent applications published prior to the filing date of this application. Any of them may be used in obtaining a preformed SiHy lens which in turn becomes the inner layer of a SiHy contact lens of the invention, so long as they will yield a SiHy material having a Dk and water content specified above. A SiHy lens formulation for making commercial SiHy lenses, such as, lotrafilcon A, lotrafilcon B, balafilcon A, galyfilcon A, senofilcon A, narafilcon A, narafilcon B, comfilcon A, enfilcon A, asmofilcon A, filcon II 3, can also be used in making preformed SiHy contact lenses (the inner layer of a SiHy contact lens of the invention). A hyaluronic acid-peptide conjugate (i.e. coating composition of the invention) may be coupled to any one of these compositions (with the exception of lotrafilcon B) and the resulting conjugate used to make a contact lens of the invention (i.e. a preformed contact lens with the coating composition of the invention disposed thereon).

The composition comprising polymerizable vinylic group and a peptide linked to a lubricant (for example hyaluronic acid), are accessible starting from commercially available hydroxyl functionalized vinylic compounds. Examples of hydroxyl functionalized vinylic compounds include and is not limited to 2-hydroxyethylmethacrylate, glyceryl methacrylate, methacrylated silicone containing hydroxyl compounds (WO2012104349) and that is covalently incorporated in to the contact lens, and wherein the peptide is cleavable by one or more proteinases present in tear fluid. Method of preparation of an example of this polymerizable composition is described in Example 4.

This above said polymerizable composition is suitable for use as a monomer formulation component for any non-silicone or preferably silicone hydrogel contact lens. Alternatively, a contact lens (preferably a silicone hydrogel contact lens) can be made according to any methods well known to a person skilled in the art. For example, contact lenses can be produced in a conventional “spin-casting mold,” as described for example in U.S. Pat. No. 3,408,429, or by the full cast-molding process in a static form, as described in U.S. Pat. Nos. 4,347,198; 5,508,317; 5,583,463; 5,789,464; and 5,849,810, or by lathe cutting of silicone hydrogel buttons as used in making customized contact lenses. In cast-molding, a lens formulation typically is dispensed into molds and cured (i.e., polymerized and/or crosslinked) in molds for making contact lenses. For production of silicone hydrogel (SiHy) contact lenses, a SiHy lens formulation for cast-molding or spin-cast molding or for making SiHy rods used in lathe-cutting of contact lenses generally comprises at least one components selected from the group consisting of a silicone-containing vinylic monomer, a silicone-containing vinylic macromer, a silicone-containing prepolymer, a hydrophilic vinylic monomer, a hydrophobic vinylic monomer, a crosslinking agent (a compound containing at least two ethylenically unsaturated groups), a free-radical initiator (photoinitiator or thermal initiator), a hydrophilic vinylic macromer/prepolymer, and combination thereof, as well known to a person skilled in the art. A SiHy contact lens formulation can also comprise other necessary components known to a person skilled in the art, such as, for example, a UV-absorbing agent, a visibility tinting agent (e.g., dyes, pigments, or mixtures thereof), antimicrobial agents (e.g., preferably silver nanoparticles), a bioactive agent, leachable lubricants, leachable tear-stabilizing agents, and mixtures thereof, as known to a person skilled in the art. Resultant SiHy contact lenses then can be subjected to extraction with an extraction solvent to remove unpolymerized components from the resultant lenses and to hydration process, as known by a person skilled in the art. In addition, a preformed SiHy contact lens can be a colored contact lens (i.e., a SiHy contact lens having at least one colored patterns printed thereon as well known to a person skilled in the art).

Lens moulds for making contact lenses are well known to a person skilled in the art and, for example, are employed in cast moulding or spin casting. For example, a mould (for cast moulding) generally comprises at least two mold sections (or portions) or mold halves, i.e. first and second mold halves. The first mould half defines a first moulding (or optical) surface and the second mould half defines a second moulding (or optical) surface. The first and second mold halves are configured to receive each other such that a lens forming cavity is formed between the first moulding surface and the second moulding surface. The moulding surface of a mould half is the cavity-forming surface of the mould and in direct contact with lens-forming material.

Methods of manufacturing mould sections for cast-molding a contact lens are generally well known to those of ordinary skill in the art. The process of the present invention is not limited to any particular method of forming a mould. In fact, any method of forming a mould can be used in the present invention. The first and second mould halves can be formed through various techniques, such as injection moulding or lathing. Examples of suitable processes for forming the mould halves are disclosed in U.S. Pat. No. 4,444,711 to Schad; U.S. Pat. No. 4,460,534 to Boehm et al.; U.S. Pat. No. 5,843,346 to Morrill; and U.S. Pat. No. 5,894,002 to Boneberger et al.

Virtually all materials known in the art for making moulds can be used to make molds for making contact lenses. For example, polymeric materials, such as polyethylene, polypropylene, polystyrene, PMMA, Topas(R) COC grade 8007-S10 (clear amorphous copolymer of ethylene and norbornene, from Ticona GmbH of Frankfurt, Germany and Summit, N.J.), or the like can be used. Other materials that allow UV light transmission could be used, such as quartz glass and sapphire.

In a preferred embodiment, reusable moulds are used and the silicone-hydrogel lens-forming composition is cured actinically under a spatial limitation of actinic radiation to form a SiHy contact lens. Examples of preferred reusable molds are those disclosed in U.S. patent application Ser. No. 08/274,942 filed Jul. 14, 1994, Ser. No. 10/732,566 filed Dec. 10, 2003, Ser. No. 10/721,913 filed Nov. 25, 2003, and U.S. Pat. No. 6,627,124, which are incorporated by reference in their entireties. Reusable moulds can be made of quartz, glass, sapphire, CaF2, a cyclic olefin copolymer (such as for example, Topas(R) COC grade 8007-S10 (clear amorphous copolymer of ethylene and norbornene) from Ticona GmbH of Frankfurt, Germany and Summit, N.J., Zeonex(R) and Zeonor(R) from Zeon Chemicals LP, Louisville, Ky.), polymethylmethacrylate (PMMA), polyoxymethylene from DuPont (Delrin), Ultem(R) (polyetherimide) from G.E. Plastics, PrimoSpire(R), etc.

The coating composition is coated onto a preformed contact lens. That is to say, the coating composition is disposed on the surface of a preformed contact lens.

The coating composition may be used in the manufacture of a contact lens of the the invention. The coating composition may be coated onto a preformed contact lens using any suitable means. At least three approaches may be envisaged:

(1) The coating composition may be connected to the surface of a preformed lens itself, such as via hydroxyl functionalities on the preformed contact lens.

(2) The hyaluronic acid-peptide conjugate (i.e. coating composition) may be added to a SiHy pre-polymer (reactive monomer formulation mix). Such formulations typically contain monomers with a free hydroxyl group and thus be coupled, for example, to the peptide carboxylic acid function of the coating composition of the invention. Following polymerization to form a preformed contact lens, the coating composition will be disposed on the surface of the preformed contact lens.

(3) Alternatively, the hyaluronic acid-peptide conjugate (i.e. coating composition may be coupled to a single component monomer and the resulting conjugate added to the SiHy pre-polymer mixture. This approach may be synthetically advantageous since no side reactions can occur with the other pre-polymer components during the chemical steps, and all steps may be monitored analytically with high precision.

In more detail, in approaches (1) or (2) a hyaluronic acid-peptide conjugate (i.e. coating composition) of the present invention may be connected to the surface of a preformed contact lens (either in the form of a fully finished lens or to a monomers/macromers/prepolymers suitable for use in the preparation of a preformed contact lens) via the C-terminal carboxylic acid or the N-terminal amino function of the peptide portion of the hyaluronic-peptide conjugate or via a side chain functionality of one of its amino acid residues, optionally via a suitable linker. Methods are well known to those skilled in the art for coupling free carboxylic acid or amino end groups on the peptide portion to functional groups present in polymers.

Contact lenses coated by a lubricant-peptide coating composition of the invention are accessible by treatment of a preformed contact lens with the lubricant-peptide conjugate (i.e. coating composition) in the presence or absence of a coupling agent to form a covalent bond or linkage under reaction conditions well known to a person skilled in the art.

The peptide portion of the lubricant-peptide conjugate may be connected to the contact lens surface using any suitable method. It is well-known by a person skilled in the art to couple chemical groups such as free acid groups, free amino groups, free hydroxyl groups on the peptides to hydroxy groups, amine groups and acid groups present on the contact lens surface.

Ester formation by reaction between carboxylic acids and alcohols is well known to those skilled on the art.

A preformed contact lens may be activated prior to treatment with the lubricant-peptide conjugate. Methods and chemistry for activation of polymeric molecules as well as for conjugation of polypeptides are intensively described in the literature. Non-limiting examples of methods described in WO 97/30148 and supporting references are used in entirety as part of this invention. Non-limiting examples include reaction between free carboxylic acid groups or free amine groups of the hyaluronic-peptide conjugate and the contact lens surface bearing oxirane function and activated hydroxyl functions may be used to form esters and amines, respectively for linking a lubricant-peptide conjugate to the contact lens surface. Additional methods of coupling chemistries that can be used employed for linking a lubricant-peptide compositions on to functional groups on the contact lens surface is described in E. S. Schante et al Carbohydrate Polymers 2011, 85, p 469-489. A contact lens according to the invention may further comprise a base coating on the preformed contact lens but beneath the coating composition. That is to say, a base coating may be disposed on the surface of the preformed contact lens underneath the coating composition, i.e. between the performed contact lens and the coating composition.

In such a contact lens, the coating composition is typically covalently attached to the base coating. The base coating typically comprises a polymeric coating material.

The text below details an alternative production route. In this route, the hyaluronic acid-peptide conjugate does not occur per se. Instead, a peptide-monomer is used to which, in a later stage, the HA is coupled.

As set out above, an alternative way to produce the lubricant-peptide-lens assembly is to first couple an N-protected peptide with its C-terminal carboxylic function to a hydroxyl group containing monomer which can be copolymerized to give the lens material. An example of such a monomer is hydroxyethyl methacrylate (HEMA). After this coupling the resulting peptide-HEMA conjugate is deprotected on the N-terminus and subsequently coupled with its free N-terminus to the lubricant (such as hyaluronic acid) using the chemistry as described above. The resulting lubricant-peptide-monomer compound is then added to the silicone pre-polymer mixture which is copolymerised with the other monomers to give the lens material. The advantage of such a method is that it is synthetically very easy since less side reactions can occur with the other pre-polymer components during the chemical steps and all steps can be monitored analytically with high precision.

A contact lens according to the invention may further comprise a plasma coating on the preformed contact lens but beneath the coating composition. That is to say, a plasma coating may be disposed on the surface of the preformed contact lens underneath the coating composition, i.e. between the performed contact lens and the coating composition.

Additional methods for linking the lubricant-peptide coating composition to a preformed contact lens include the incorporation of a base coating disposed on the surface of the preformed contact lens underneath the coating composition, i.e. between the performed contact lens and the coating composition.

The base may comprise a crosslinked polymer containing cyclic heterocyclic rings that are prone to ring opening by the free carboxylic acid or free amine functional groups present in the hyaluronic-peptide coating composition. Non-limiting examples include epoxides, cyclic carbonate, and positively charged azetidinium groups that react with functional groups such as alcohol, amine, and carboxylate groups to form covalent linkages. US20040236119 and US20050113594 describe coupling chemistries on cyclic carbonates. Methods of crosslinking using azetidinium groups are described in US55100014, US2011/071791A1, WO2012/016098 A1, and US2013/0148077 A1,

Plasma technology is one of the key technologies for the production of functional surfaces. Hydroxyl, amino, and carboxyl groups that are generated on the surface of the contact lens by plasma technology can be used to deposit the lubricant-peptide coating composition. Methods for the generation of chemically reactive surfaces are described by K. S. Siow et al Plasma Process and Polymers 2006, 3, p 392-418.

The invention further provides a method for the manufacture of a coating composition which method comprises linking a peptide to a lubricant (as described herein), wherein the peptide is cleavable by one or more proteinases present in tear fluid. Optionally, the resulting peptide-lubricant conjugate may be linked to a monomer, macromer or prepolymer suitable for use in the manufacture of a contact lens.

The invention also provides a method for the manufacture of a contact lens which method comprises: providing a preformed contact lens; and coating said contact lens with a coating composition of the invention.

Where the coating composition is linked to a monomer, macromer or prepolymer suitable for use in the manufacture of a contact lens, the invention provides a method for the manufacture of a contact lens which method comprises preparing a preformed contact lens in the presence of such a coating composition. That is to say, the method may comprise preparing a contact lens using any suitable material, i.e. contact-lens forming material, to which the said coating composition has been added.

A contact lens according to the invention may be comprised within a packaging. Lens packages (or containers) are well known to a person skilled in the art for autoclaving and storing a soft contact lens. Any lens packages can be used in the invention. Preferably, a lens package is a blister package which comprises a base and a cover, wherein the cover is detachably sealed to the base, wherein the base includes a cavity for receiving a sterile packaging solution and the contact lens.

Contact lenses are typically packaged in individual packages, sealed, and sterilized (e.g., by autoclave at about 120° C. or higher for at least 30 minutes) prior to dispensing to users. A person skilled in the art will understand well how to seal and sterilize lens packages.

In accordance with the invention, a packaging solution may contain at least one buffering agent and one or more other ingredients known to a person skilled in the art. Examples of other ingredients include without limitation, tonicity agents, surfactants, antibacterial agents, preservatives, and lubricants (or water-soluble viscosity builders) (e.g., cellulose derivatives, polyvinyl alcohol, polyvinyl pyrrolidone).

The packaging solution may contain a buffering agent in an amount sufficient to maintain a pH of the packaging solution in the desired range, for example, preferably in a physiologically acceptable range of about 6 to about 8.5. Any known, physiologically compatible buffering agents can be used. Suitable buffering agents as a constituent of the contact lens care composition according to the invention are known to the person skilled in the art. Examples are boric acid, borates, e.g. sodium borate, citric acid, citrates, e.g. potassium citrate, bicarbonates, e.g. sodium bicarbonate, TRIS (2-amino-2-hydroxymethyl-1,3-propanediol), Bis-Tris(Bis-(2-hydroxyethyl)-imino-tris-(hydroxymethyl)-methane), bis-aminopolyols, triethanolamine, ACES (N-(2-hydroxyethyl)-2-aminoethanesulfonic acid), BES (N,N-Bis(2-hydroxyethyl)-2-aminoethanesulfonic acid), HEPES (4-(2-hydroxyethyl)-1-piperazineethanesulfonic acid), MES (2-(N-morpholino)ethanesulfonic acid), MOPS (3-[N-morpholino]-propanesulfonic acid), PIPES (piperazine-N,N′-bis(2-ethanesulfonic acid), TES (N-[Tris(hydroxymethyl)methyl]-2-aminoethanesulfonic acid), salts thereof, phosphate buffers, e.g. Na2HPO4, NaH2PO4, and KH2PO4 or mixtures thereof. A preferred bis-aminopolyol is 1,3-bis(tris[hydroxymethyl]-methylamino)propane (bis-TRIS-propane). The amount of each buffer agent in a packaging solution is preferably from 0.001% to 2%, preferably from 0.01% to 1%; most preferably from about 0.05% to about 0.30% by weight.

The packaging solution typically has a tonicity of from about 200 to about 450 milliosmol (mOsm), preferably from about 250 to about 350 mOsm. The tonicity of a packaging solution can be adjusted by adding organic or inorganic substances which affect the tonicity. Suitable occularly acceptable tonicity agents include, but are not limited to sodium chloride, potassium chloride, glycerol, propylene glycol, polyols, mannitols, sorbitol, xylitol and mixtures thereof.

A packaging solution of the invention typically has a viscosity of from about 1 centipoise to about 20 centipoises, preferably from about 1.5 centipoises to about 10 centipoises, more preferably from about 2 centipoises to about 5 centipoises, at 25° C.

In a preferred embodiment, the packaging solution comprises preferably from about 0.01% to about 2%, more preferably from about 0.05% to about 1.5%, even more preferably from about 0.1% to about 1%, most preferably from about 0.2% to about 0.5%, by weight of a water-soluble and thermally-crosslinkable hydrophilic polymeric material for forming the top coating.

Where at least one of the crosslinked coating and the packaging solution contains a polymeric material having polyethylene glycol segments, the packaging solution preferably comprises an [alpha]-oxo-multi-acid or salt thereof in an amount sufficient to have a reduced susceptibility to oxidation degradation of the polyethylene glycol segments. A commonly-owned co-pending patent application (US patent application publication No. 2004/0116564 A1, incorporated herein in its entirety) discloses that oxo-multi-acid or salt thereof can reduce the susceptibility to oxidative degradation of a PEG-containing polymeric material.

Exemplary α-oxo-multi-acids or biocompatible salts thereof include without limitation citric acid, 2-ketoglutaric acid, or malic acid or biocompatible (preferably ophthalmically compatible) salts thereof. More preferably, an α-oxo-multi-acid is citric or malic acid or biocompatible (preferably ophthalmically compatible) salts thereof (e.g., sodium, potassium, or the like).

In accordance with the invention, the packaging solution can further comprise mucin-like materials (e.g., polyglycolic acid, polylactides, and the likes), ophthalmically beneficial materials (e.g., 2-pyrrolidone-5-carboxylic acid (PCA), glycolic acid, lactic acid, malic acid, tartaric acid, mandelic acid, citric acids, linoleic and gamma linoleic acids, salts thereof, taurine, glycine, and vitamins), and/or surfactants.

Where the contact lens of the invention comprises a preformed silicone hydrogel contact lens, the contact lens preferably has at least one of the properties selected from the group consisting of:

an oxygen permeability of at least about 40 barrers, preferably at least about 50 barrers, more preferably at least about 60 barrers, even more preferably at least about 70 barrers;

an elastic modulus of about 1.5 MPa or less, preferably about 1.2 MPa or less, more preferably about 1.0 or less, even more preferably from about 0.3 MPa to about 1.0 MPa;

an Ionoflux Diffusion Coefficient, D, of, preferably at least about 1.5×10⁻⁶ mm²/min, more preferably at least about 2.6×10⁻⁶ mm²/min, even more preferably at least about 6.4×10⁻⁶ mm²/min; a water content of preferably from about 18% to about 70%, more preferably from about 20% to about 60% by weight when fully hydrated; or combinations thereof.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

### Embodiments of the Invention

1. A composition comprising a peptide linked to a lubricant, wherein the peptide is cleavable by one or more proteinases present in tear fluid.
2. A composition according to embodiment 1 which is a coating composition.
3. A composition according to embodiment 1 or 2, wherein the lubricant is hyaluronic acid, a cellulose derivative, a dextran, a polymeric alcohol, a polyvinyl alcohol or povidone (polyvinylpyrrolidone).
4. A composition according to any one of the preceding embodiments, wherein the lubricant has a molecular weight of between 200 Da and 2 MDa.
5. A composition according to embodiment any one of the preceding embodiments, wherein the peptide linker is cleavable by a serine proteinase or a metalloproteinase present in tear fluid
6. A composition according to any one of the preceding embodiments wherein the peptide comprises any one of the amino acid residues Ala, Ile, Leu, Phe, Asn, Gln, Pro, Gly or Val.
7. A composition according to any one of the preceding embodiments wherein the peptide comprises any one of the amino acid residues of Ala, Leu, Gln, Pro and Gly.
8. A composition according to any one of the preceding embodiments which comprises the amino acid sequence Leu-Ala-Leu-Leu-Ala (SEQ ID NO: 1) or Leu-Leu-Leu-Ala-Ala-Gly (SEQ ID NO: 6).
9. A composition according to any one of the preceding embodiments which is linked to a monomer, macromer or prepolymer suitable for use in the manufacture of a contact lens.
10. A composition of according to embodiment 9, wherein the composition comprises a polymerizable vinylic group.
11. A contact lens or ocular implant that comes into contact with tear fluid comprising a composition according to any one of the preceding embodiments.
12. A contact lens or ocular implant that comes into contact with tear fluid comprising a preformed contact lens and:
   a composition according to any one of embodiments 1 to 8 coated thereon; or
   a composition according to embodiment 9 or 10.
13. A contact lens according to embodiment 11 or 12, wherein the preformed contact lens is composed of a hydrogel material.
14. A contact lens according to embodiment 13, wherein the contact lens is a silicone hydrogel contact lens comprising a silicone hydrogel material.
15. A contact lens according to any one of embodiments 11 to 14 further comprising a base coating on the preformed contact lens but beneath the composition according to any one of embodiments 1 to 8.
16. A contact lens according to embodiment 15, wherein the composition is covalently attached to the base coating.
17. A contact lens according to embodiment 15 or 16, wherein the base coating comprises a polymeric coating material.
18. A contact lens according to any one of embodiments 12 to 17 further comprising a plasma coating on the preformed contact lens but beneath the composition according to any one of embodiments 1 to 8.
19. A contact lens according to any one of embodiments 11 to 18 which is a disposable contact lens.
20. A method for the manufacture of a composition which method comprises linking a peptide to a lubricant, wherein the peptide is cleavable by one or more proteinases present in tear fluid and, optionally, linking the resulting peptide-lubricant conjugate to a monomer, macromer or prepolymer suitable for use in the manufacture of a contact lens.
21. A method for the manufacture of a composition which method comprises linking a peptide to a monomer, macromer or prepolymer suitable for use in the manufacture of a contact lens, wherein the peptide is cleavable by one or more proteinases present in tear fluid and linking the resulting peptide-monomer, -macromer or -prepolymer conjugate to a lubricant.
22. A method according to embodiment 20 or 21, wherein the lubricant is hyaluronic acid, a cellulose derivative, a dextran, a polymeric alcohol, a polyvinyl alcohol or povidone (polyvinylpyrrolidone)
23. A method for the manufacture of a contact lens which method comprises:
   providing a preformed contact lens; and
   coating said contact lens with a composition according to any one of embodiments 1 to 8.
24. A method for the manufacture of a contact lens which method comprises:
   preparing a preformed contact lens in the presence of a composition according to embodiment 9 or 10.
25. A method for the manufacture of a contact lens which method comprises:
   preparing a composition according to any one of embodiments 20 to 22 and preparing a preformed lens in the presence of the resulting composition.
26. A composition comprising a monomer, macromer or prepolymer suitable for use in the manufacture of a contact lens and a peptide cleavable by one or more proteinases present in tear fluid.
27. A composition according to embodiment 26, wherein the monomer, macromer or prepolymer comprises a polymerizable vinylic group.
28. A contact lens or ocular implant that comes into contact with tear fluid comprising a composition according to embodiment 26 or 27.
29. A contact lens according to embodiment 28, wherein a lubricant is linked to the contact lens through the peptide.
30. A method for the manufacture of a contact lens which method comprises:
   preparing a preformed contact lens in the presence of a composition according to embodiment 26 or 27 and;
   linking a lubricant to the thus formed preformed contact lens via the peptide.

The present invention is further illustrated by the following Examples:

### EXAMPLES

### Materials and Methods

### Peptide Synthesis and Purification

The peptides were synthesized using standard solid phase techniques (W. C. Chan, P. D. White, Fmoc solid phase peptide synthesis: a practical approach, Oxford University Press, 2000). After cleavage from the resin with trifluoroacetic acid the peptide was precipitated from solution with MTBE/heptane and subsequently lyophilized. The peptides were further purified by preparative HPLC on a Varian PrepStar system using a stationary-phase column (Pursuit XRs, C18, 10 mm particle size, 500×41.4 mm internal diameter) at room temperature. UV detection was performed at 220 nm and 254 nm using a UV-VIS Varian ProStar spectrometer. The gradient program was: 0-25 min linear gradient from 5% to 95% eluent B and from 25.1-30 min 5% eluent B (eluent A: 1 mL/L formic acid in H₂O; eluent B: 1 mL/L formic acid in CH₃CN), with a flow rate of 50 mL/min. Injection volumes were 10 mL. Pure fractions were pooled and lyophilized. Lyophilization was performed on a VaCo 5 (II) lyophilizer from Zirbus technologies.

### Mass Spectrometry

Stock solutions (1 mg/ml in 5% acetonitrile) were prepared from the synthetic peptide substrates. The stock solutions are optionally 10× diluted with MilliQ water prior to incubation. Elastase (Sigma #: E7885), Tryptase (Sigma #: T7063), or tear fluid were added to the diluted substrate solutions and incubation was performed at 25° C. Aliquots of 20 μl were taken at several time points to monitor substrate degradation.

The aliquots were 10× diluted in 50% acetonitrile, 0.1% formic acid prior to analysis. Mass Spectrometric (MS) analysis was performed by infusion in the LTQ-Orbitrap Fourier Transform Mass Spectrometer (Thermo Fisher, Bremen, Germany). Infusion was performed by mixing 10 μl min⁻¹ of sample in a 200 μl min⁻¹ flow of 50% acetonitrile, 0.1% formic acid. The MS analysis was performed in the Orbitrap scanning at 7500 resolution using 200-1600 m/z mass range. Proteolytic activity during the different incubations of the synthetic peptides substrates was studied by manual inspection of the MS data using the Qual browser in the XCalibur software (Thermo Fisher, Bremen, Germany).

### Example 1

### Proteases in Tear Liquid

According to the literature, inflammation reactions, allergen exposure or physical contacts may provoke proteolytic activity in tear fluid. Such a proteolytic activity would be applicable in cleaving short peptide linkers between an eye lubricant, e.g. hyaluronic acid, and the surface of a contact lens. In view of the, presumably minimal, proteolytic activity generated, it is of utmost importance that the amino acid sequence of the peptide linker provides the optimal cleavage site for the relevant proteolytic activity. In the present Example we analyze the nature of the proteolytic activity present on the contact lens surface of nine individuals. Two individuals were wearing hard lenses, all the others were using soft lenses. The wearing period of these soft lenses varied between one day and two years.

Contact lens wearing subjects were invited to participate at the end of their working day. Using sterilized gloves, lenses were removed and the inner surface of each lens was rinsed with 250 microliter of sterilized water. Then 100 microliter samples of the rinsing liquid were incubated overnight at 25 degrees C. with 100 μl of a 1.0 mg/ml BODIPY TR-X casein solution (EnzChek Protease Assay kit ‘Red fluorescence’; Molecular Probes, Eugene, Oreg., USA). Throughout the incubation the fluorescence of the samples was measured every minute (kinetic measurement) according to the EnzChek kit protocol (excitation=590±10 nm, emission=645±20 nm) using a Tecan Infinite M1000 microtiterplate reader (Männedorf, Switzerland). The results obtained showed that the contact lens rinsing liquid of all participants exhibited proteolytic activity, although there were some differences between subjects in the levels of proteolytic activity present.

Subsequently the experiment was repeated but this time with the aim of identifying the nature of the proteolytic activities present. To do this, three different selective protease inhibitors were added to the contact lens rinsing liquid, EDTA to inhibit metallo endopeptidases (IUBMB enzyme class EC3.4.24), PMSF to inhibit serine endopeptidases (IUBMB enzyme class EC3.4.21) and E64 to inhibit cysteine endopeptidases (IUBMB enzyme class EC3.4.22). Because of their very acid pH optima, a significant proteolytic activity of aspartic endoproteases (IUBMB enzyme class EC3.4.23) was seen as unlikely. EDTA ((Merck, Darmstadt, Germany) was used in a final concentration of 5 millimol/l, PMSF ((Molekula, Munchen, Germany) was used in a final concentration of 1 millimol/l and E-64 (Sigma-Aldrich) in a final concentration of 10 micromol/l. As before, the rinsing liquid with the various inhibitors added were incubated overnight with the Enzcheck Protease kit and the next morning proteolytic activities were measured. According to the results obtained, most rinsing liquids incorporated serine as well as metallo endopeptidase activity. In a single case only metallo endopeptidase activity was recorded. Cysteine endopeptidase activity was never present.

### Example 2

### Proteases in Tear Liquid can Cleave Specific Peptides

In tear fluid 491 different proteins have been identified (de Souza et al., Genome Biology 2006, 7: R72) and among these 32 different proteinases. The following classes of endopeptidases occur: Metallo peptidases (a.o. matrix metallo proteinases and stromelysins), serine peptidases (myeloblastine, leukocyt elastase, tryptase, plasminogeen, prostacine, and cathepsine G), cysteine proteinases (cathepsine B and cathepsine Z) and aspartyl proteinases (cathepsine D).

In Example 1, it is shown that, in tear liquid, only two of these four classes of endopeptidases are responsible for the majority of the proteolytic activity present, that is metallo peptidases and serine peptidases. Therefore, to demonstrate peptide cleavage by tear fluid, the test peptide should present an amino acid sequence that is cleavable by representatives of both classes of endopeptidases. Moreover, amino acids with reactive side groups complicating the chemical conjugation of the peptide to the lubricant of choice or to the contact lens surface should be avoided. Bearing this in mind, the peptide Gly-Pro-Leu-Ala-Leu-Leu-Ala-Gln (GPLALLAQ) (SEQ ID NO: 2) was synthesized.

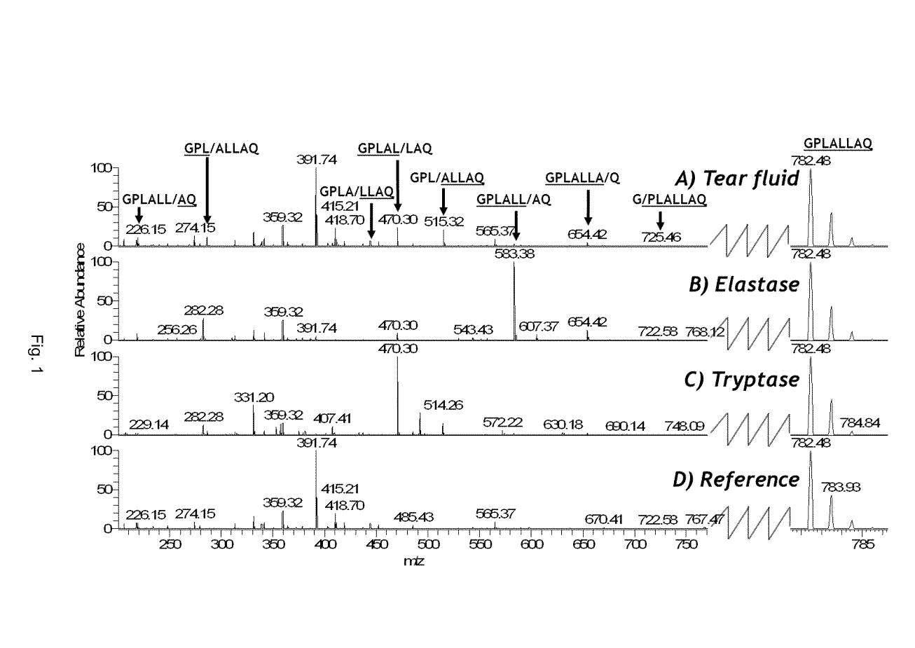

After its purification, the peptide was incubated with contact lens rinsing liquid (cf. Example 1) of a single individual and incubated over the weekend at 25° C. Then samples of the incubation liquid were subjected to mass spectrometry to confirm cleavage. According to the results, cleavage products identified include GPLAL and LAQ, GPL and ALLAQ and GPLA and LLAQ. An incubation of the peptide with human leukocyte elastase (Sigma Aldrich) yielded GPLALL and AQ as the major cleavage products. An incubation of the peptide with human lung tryptase (also from Sigma Aldrich) yielded GPLAL/LAQ as the major cleavage products (see FIG. 1). The latter observations indicate that a peptide incorporating aliphatic hydrophobic residues like Ala and Leu support cleavage by human, tryptase- and elastase-like enzymes.

### Example 3

### Preparation of a Polysaccharide-Peptide Conjugate Via Reducing Terminal Residue/Limited Oxidation Method

To demonstrate the feasibility of coupling a suitable lubricant compound with a peptide, a conjugate of hyaluronic acid with a Gly-Tyr-OH dipeptide was prepared.

### A. Reduction

Hyaluronic acid (Hyasis, Novozymes (China) Biopharma Co., Ltd) was treated with aq. HCl (see K. Tømmeraas, Biomacromolecules 2008, 9, 1535-1540) to reduce its molecular weight to approximately 10.000 Da and 5 g was dissolved in 100 mL of water and the pH of the resulting solution was adjusted to 5 using 1N NaOH. Subsequently, NaBH₄ (0.3 g; 8 mmol) was added and the pH adjusted to 8-9 through the addition of acetic acid. The reaction mixture was stirred for 5 hours at ambient temperature and subsequently concentrated in vacuo to a volume of approximately 10 mL. Ethanol (150 mL) was added and the precipitated product was isolated by filtration. Yield 5.3 g white solid.

### B. Oxidation

The reduced hyaluronic acid prepared in the preceding step (5.3 g; 0.5 mmol) was dissolved in 100 mL of water and subsequently NaIO₄ (0.5 g; 2.5 mmol) was added. The reaction mixture was stirred for one hour at 20° C. and then concentrated in vacuo to a volume of ˜10 mL. Ethanol (150 mL) was added to the reaction mixture and the precipitated product was isolated through filtration. Isolated yield: 4.7 g. ¹H-NMR analysis confirmed the formation of the desired aldehyde.

### C. Reductive Amination

The aldehyde prepared in the previous step (4.7 g; 0.5 mmol) was dissolved in 100 mL 0.05 M borate buffer (pH=8.5). To this solution was added the dipeptide Gly-Tyr-OH (0.23 g; 1 mmol) followed by NaCNBH₃ (0.2 g; 3.2 mmol). The reaction mixture was stirred for 60 hours at ambient temperature and subsequently concentrated in vacuo. Ethanol (100 mL) was added to the resulting wet solid material and subsequently the crude product was isolated through filtration. Excess dipeptide and inorganic salts were removed using a dialysis membrane (cut off 3.5 kDa) and the purified product was isolated through lyophilization. The product was characterized by 400 MHz ¹H-NMR. The absence of un-coupled dipeptide was demonstrated through HPLC-analysis.

### Example 4

### Preparation of a Polysaccharide-Peptide-Lens Monomer Conjugate

To demonstrate the feasibility of coupling a peptide to a lens monomer and subsequently coupling this peptide-lens monomer conjugate to a suitable lubricant compound, a conjugate of hyaluronic acid with an Ala-Leu-Ala-Leu (SEQ ID NO: 3) tetrapeptide and HEMA (hydroxylethyl methacrylate) is prepared.

### A. Preparation of Ala-Leu-Ala-Leu-HEMA

Fmoc-Ala-Leu-Ala-Leu is prepared using standard solid-phase peptide synthesis protocols (see Fmoc Solid Phase Peptide Synthesis by W. C. Chan and P. D. White, Oxford university press, 2004). Fmoc-Ala-Leu-Ala-Leu (2 mmol) is reacted with HEMA (2 mmol) and dicyclohexyl carbodiimide (DCC) in 20 mL of dichloromethane until the reaction reaches completion (HPLC). After the reaction, the Fmoc-Ala-Leu-Ala-Leu is purified by precipitation or chromatography. Subsequently, the Fmoc group may be cleaved by treatment with an organic base and the resulting Ala-Leu-Ala-Leu-HEMA isolated and purified by chromatography.

### B. Reductive Amination

The hyaluronic aldehyde prepared in step B of Example 3 (4.7 g; 0.5 mmol) is dissolved in 100 mL 0.05 M borate buffer (pH=8.5). To this solution is added Ala-Leu-Ala-Leu-HEMA (1 mmol) followed by NaCNBH₃ (0.2 g; 3.2 mmol). The reaction mixture is stirred for 60 hours at ambient temperature and subsequently concentrated in vacuo. Ethanol (100 mL) is added to the resulting wet solid material and subsequently the crude product is isolated through filtration. Excess Ala-Leu-Ala-Leu-HEMA and inorganic salts are removed using a dialysis membrane and the purified product is isolated through lyophilization. The product is characterized by 400 MHz 1H-NMR.

### Example 5

### Cleavage of Other Synthetic Peptides by Tear Fluid

As demonstrated in Example 1, metallo endopeptidases as well as serine endopeptidases can be active in tear fluids. In the human cornea, so called matrix metalloproteinases (MMP's) are secreted by epithelial cells, stromal cells and neutrophils. A.o MMP's 1, 2, 8, 9 and 13 have been detected in tear fluid (de Souza et al., Genome Biology 2006, 7:R72; Ollivier et al., Veterinary Ophthalmology (2007) 10, 4, 199-206; Balasubramanian et al., Clin Exp Optom 2013; 96:214-218; Zhou et al., Journal of Proteomics 75 (2012)3877-3885). Noteworthy are the elevated levels of MMP-9 recorded for individuals suffering from dry eyes (Acera et al., Ophthalmic Res 2008; 40(6):315-321). The various MMP's are known to preferably cleave peptide bonds involving hydrophobic amino acids such as Ala, Leu and Phe but their substrate specificity seems to be conferred at much broader positions so that cleavage by a particular MMP is hard to predict.

Additionally serine endoproteases have been detected in tear fluid. The latter group of endoproteases can be subdivided in trypsin-like and elastase-like activities. The trypsin-like endoprotease tryptase is released by mast cells (Butrus et al., Ophthalmology, 1990, Vol 97, No 12, pp 1678-1683) and is known to favor cleavage of peptide bonds involving charged basic residues like Arg or Lys. The elastase-like activity includes leukocyte elastase and myeloblastin (de Souza et al., Genome Biology 2006, 7:R72) which are known to favor cleavage of peptide bonds after small aliphatic residues like Ala, Val and Ser but cleavage after larger residues like Ile and Leu is also possible.

In this Example we test the cleavage activity of tear fluid on two, slightly different synthetic peptides. The first peptide comprises Arg as a charged, basic residue (Ala-Ala-Pro-Arg-Ala-Ala-Arg-Gln, AAPRAARQ (SEQ ID NO: 4)) in order to facilitate cleavage by trypsin-like endoproteases. The second peptide comprises the medium sized aliphatic hydrophobic residue Val instead of Arg (Ala-Ala-Pro-Val-Ala-Ala-Arg-Gln, AAPVAARQ (SEQ ID NO: 5)) in order to facilitate cleavage by elastase-like endoproteases. The experiment was executed essentially as described in Example 2, but in this case tear fluid of four different individuals was obtained and incubated for 165 hours at 25 degrees C. Samples of the incubation liquid were again subjected to mass spectrometry.

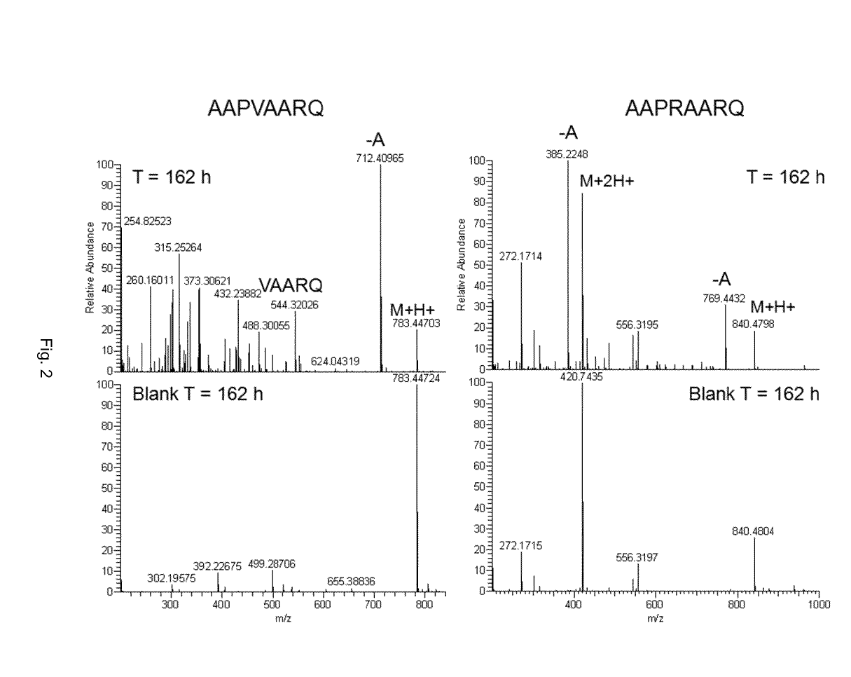

Quite unexpectedly, none of the two peptides yielded a fragment that could be traced back to an endoproteolytic cleavage after Arg or Val. The implication is that apparently charged, basic residues like Arg and Lys or the medium sized aliphatic hydrophobic residue Val are less relevant for supporting rapid peptide cleavage by tear fluid. Nonetheless both peptides are sensitive to proteolytic degradation. As shown in FIG. 2, some cleavage of the Pro-Val peptide bond does occur indicating the activity of a proline-specific endoprotease. Additionally, the data show that from both peptides used, the N-terminal Ala residue is removed during incubation. Such an exoproteolytic activity suggests the presence of an aminopeptidase activity (EC 3.4.11) in the tear liquid. However, this activity is of no use for the present invention as in a lubricant-peptide-monomer conjugate the peptide's amino-terminus is blocked by the lubricant.

### Example 6

### Preparation of a Polysaccharide-Peptide-Lens Monomer Conjugate

To demonstrate the feasibility of coupling a peptide to a lens monomer and subsequently coupling this peptide-lens monomer conjugate to a suitable lubricant compound, a conjugate of hyaluronic acid with a NH₂-Leu-Leu-Leu-Ala-Ala-Gly (SEQ ID NO: 6) hexapeptide and HEMA (hydroxyethyl methacrylate) was prepared.

### A. Preparation of Leu-Leu-Leu-Ala-Ala-Gly-HEMA

Fmoc-Leu-Leu-Leu-Ala-Ala-Gly was prepared using standard solid-phase peptide synthesis protocols (see Fmoc Solid Phase Peptide Synthesis by W. C. Chan and P. D. White, Oxford university press, 2004). A solution of Fmoc-Leu-Leu-Leu-Ala-Ala-Gly (2.0 g, 2.6 mmol), HEMA (3.20 mL, 25.0 mmol), 4-dimethylaminopyridine (DMAP, 0.04 g, 0.3 mmol) and dicyclohexyl carbodiimide (DCC, 0.60 g, 2.9 mmol) in 40 mL of N,N-dimethyl formamide (DMF) was stirred at 0° C. for 1 h and another 16 h at ambient temperature. During that time the reaction had reached completion (HPLC, full conversion of Fmoc-Leu-Leu-Leu-Ala-Ala-Gly). Subsequently, piperidine (4.0 mL) was added and the reaction mixture was stirred for another 1 h at ambient temperature. The resulting reaction mixture was poured, under vigorous stirring, into 160 mL n-heptane/methyl-tert-butyl ether 1:1 (v/v), resulting in precipitation of the product. This product was centrifuged and the resulting solid was stirred vigorously in 160 mL n-heptane/methyl-tert-butyl ether 1:1 (v/v) and centrifuged once more. The resulting solid was dissolved in 20 mL acetonitrile/water 4:1 (v/v) and freeze dried, giving 1.0 g of crude NH₂-Leu-Leu-Leu-Ala-Ala-Gly-HEMA, which was purified by preparative HPLC, giving 0.11 g of pure NH₂-Leu-Leu-Leu-Ala-Ala-Gly-HEMA. ¹H-NMR confirmed the identity of the compound; no other components were visible.

### B. Reductive Amination

The purified NH₂-Leu-Leu-Leu-Ala-Ala-Gly-HEMA (50 mg, 0.07 mmol) was dissolved in a mixture of 10 mL THF and 10 mL 0.05 M borate buffer (pH=8.5). The hyaluronic aldehyde as prepared in step B of Example 3 (0.5 g; 0.05 mmol) was added to this mixture followed by NaCNBH₄ (40 mg; 0.6 mmol). The reaction mixture was stirred for 72 hours at ambient temperature and subsequently concentrated in vacuo. Ethanol (100 mL) was added to the resulting wet solid material and subsequently the product was isolated through filtration. LC-MS analysis indicated that no NH₂-Leu-Leu-Leu-Ala-Ala-Gly-HEMA was present in the resulting product. The product was characterized by 300 MHz ¹H-NMR (DSMO-d₆). By integration of the metacrylate protons (at 6.1 and 5.7 ppm) and comparison with the hyaluronic CH₃-acetyl protons (1.92 ppm) it was estimated that the resulting product consisted of 40% hyaluronic acid-peptide-HEMA conjugate and 60% unreacted hyaluronic acid.

### Example 7

### Tear Fluid Mediated Release of Hyaluronic Acid from the Hyaluronic Acid-Peptide-Monomer Conjugate

As a consequence of the large and heterologous size of the hyaluronic acid moiety of the conjugate, its release by means of peptide hydrolysis is difficult to demonstrate. To cope with this experimental difficulty, we decided to focus on the identification of Peptide-HEMA fragments that can be expected to be released from the hyaluronic acid-peptide-HEMA conjugate upon exposure to a suitable proteolytic activity. On the basis of peptide hydrolysis data gathered in Examples 2 and 5, peptide Leu-Leu-Leu-Ala-Ala-Gly (LLLAAG) was synthesized and used to prepare a hyaluronic acid-peptide-HEMA conjugate (Example 6). The resulting conjugate was then incubated with the human elastase preparation (cf. Example 2) and with lyophilized contact lens rinsing liquid of five individuals with the aim of demonstrating the formation of Peptide-HEMA fragments.

### Experimental

### Collecting of Rinsing Liquid

Rinsing liquid was collected from 5 individuals, two wearing hard lenses and three wearing soft lenses, at the end of the working day over multiple days. The lenses were collected using sterile nitrile gloves and rinsed with 200 μl MilliQ water on a flat glass microscope slide (Thermo Scientific) containing Grace Bio-labs Press-to-Seal silicone isolator, No PSA (Sigma Aldrich GBL664504-25EA). The rinse liquid was transferred to a Protein LoBind Tube 2.0 ml (Eppendorf) using Maxymum Recovery pipette tips (Axygen scientific). The rinse liquid was frozen at −80° C. as soon as possible. Rinsing fluid was lyophilized overnight when >1.5 ml rinsing liquid was collected for each individual.

### Testing Cleavage of the Pure LLLAAG Peptide by Elastase

The LLLAAG peptide was dissolved in 50 mM ammonium acetate buffer 1 mg ml⁻¹. This solution was 100× diluted in 50 mM ammonium acetate buffer. Elastase (E7885-5 mg, Sigma Aldrich) was dissolved in 50 mM ammonium acetate buffer 0.3 mg ml⁻¹. 10 μl of this elastase solution was added to 500 μl diluted peptide solution. The sample was incubated overnight at room temperature and then subjected to mass spectrometric (MS) analysis.

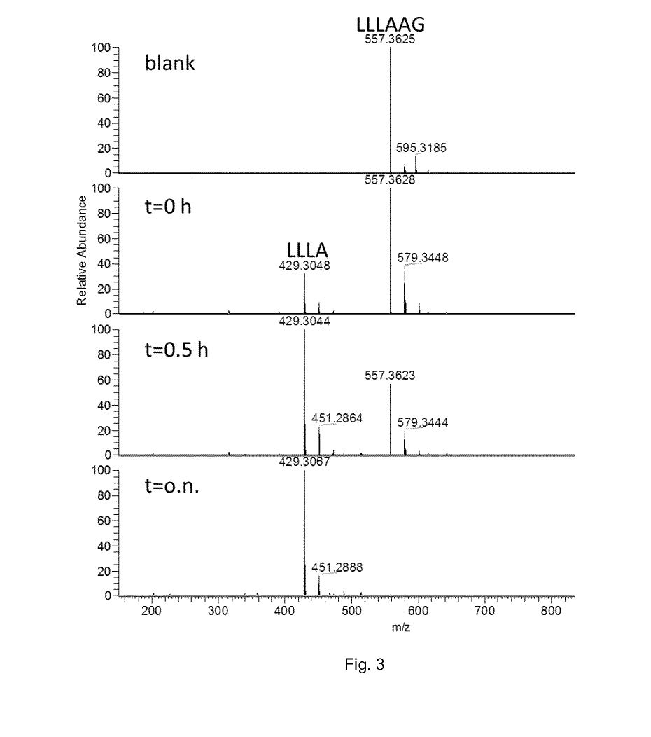

MS analysis was performed in time by 10 μl min⁻¹ infusion of 100 μl aliquots of the sample in the LTQ-Orbitrap Fourier Transform Mass Spectrometer (Thermo Fisher, Bremen, Germany). The MS analysis was performed in the Orbitrap scanning at 7500 resolution using 150-2000 m/z mass range, the instrument was calibrated prior to each experiment, ensuring accurate mass measurements <2 ppm mass accuracy. The cleavage was studied by manual inspection of the MS data using the Qual browser in the XCalibur software (Thermo Fisher, Bremen, Germany). The data obtained show that in the blank incubation only the intact precursor peptide could be detected (FIG. 3). In the incubation with elastase precursor peptide Leu-Leu-Leu-Ala-Ala-Gly (LLLAAG) was converted to fragment Leu-Leu-Leu-Ala (LLLA) hereby confirming that elastase is able to cleave the newly designed peptide.

### LC-MS Analysis of the Conjugate Products

Hyaluronic acid-peptide-HEMA conjugates are too heterologous in mass to be directly detected by MS. Also, the detection of the peptide and peptide fragments is troubled in the presence of the conjugate due to suppression effects. Therefore an LC-MS method had to be developed to enable separation of the remaining conjugate and the formed peptide-HEMA fragments.

The conjugate (Example 6) was dissolved in 50 mM ammonium acetate buffer at 0.2 mg/ml and analyzed by 25 μl injections on the LC-MS system. The LC-MS system consisted of an Accela UHPLC (Thermo Fisher, Bremen, Germany) coupled to the LTQ-Orbitrap Fourier Transform Mass Spectrometer. The column used was a ZORBAX Rapid Resolution HT SB-C18, 2.1×50 mm, 1.8 μm (Agilent 827700-902). The autosampler was set to 35° C. for incubation of the samples and the column oven was set to 50° C. The analytes in the samples were separated using a flow of 0.8 ml min⁻¹ and the following gradient was used:

0-0.2 min 2.5% B, 0.2-2 min 2.5-30% B, 2-2.5 min 30-80% B, 2.5-3 min 80% B, 3.01-5 min 2.5% B, where buffer A is 0.1% Formic Acid in Water (Biosolve, LC-MS grade) and buffer B is 0.1% Formic Acid in Acetonitrile (Biosolve, LC-MS grade). The MS was scanning at 7500 resolution m/z 150-800. Incubations were performed in the autosampler and blank injections were performed between each run. A blank incubation of conjugate without any contact lens rinsing liquid was taken along as reference, as well as an incubation of the conjugate with the human elastase preparation.

Incubating the conjugate with elastase showed the formation of the expected AG-HEMA fragment, AG being the complementary peptide part to the above mentioned Leu-Leu-Leu-Ala (LLLA) fragment (data not shown).

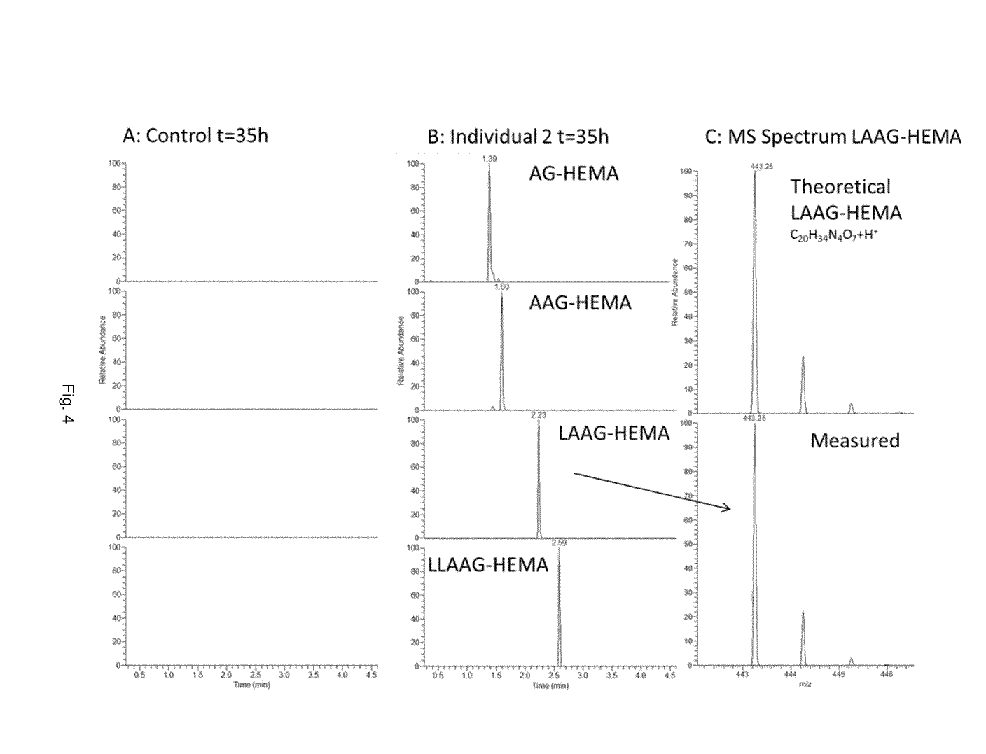

Incubations of the conjugate with the lyophilized contact lens rinsing liquid resulted in the formation of various peptide-HEMA fragments. FIG. 4, Panel B illustrates the monitoring of the peptide-HEMA fragments generated by the contact lens rinsing liquid of a single individual based on their accurate mass. MS/MS experiments were performed to confirm the identity of these peptide-HEMA fragments. A blank incubation of the conjugate without lyophilized contact lens rinsing liquid performed in parallel indicated the absence of any peptide-HEMA fragments (FIG. 4, Panel A) hereby demonstrating the need for rinsing liquid to form such fragments. Important to note is that the rinsing liquid of the other four individuals resulted in the formation of a set of peptide-HEMA fragments identical to the one formed by the rinsing liquid of the first individual, only the ratio of the various peptide fragments varied among them.

Together these data show that contact lens rinsing liquid, obtained from either soft or hard contact lenses, is able to cleave the peptide of the hyaluronic acid-peptide-HEMA conjugate. The consequence of this peptide cleavage is that hyaluronic acid is liberated from the conjugate so that it becomes freely available in the tear fluid where it will help to relieve eye discomfort.

1. A composition comprising a peptide linked to a lubricant, wherein the peptide is cleavable by one or more proteinases present in tear fluid.

2. A composition according to claim 1 which is a coating composition.

3. A composition according to claim 1, wherein the lubricant is hyaluronic acid, a cellulose derivative, a dextran, a polymeric alcohol, a polyvinyl alcohol or povidone (polyvinylpyrrolidone).

4. A composition according to claim 1, wherein the lubricant has a molecular weight of between 200 Da and 2 MDa.

5. A composition according to claim 1, wherein the peptide linker is cleavable by a serine proteinase or a metalloproteinase present in tear fluid

6. A composition according to claim 1 wherein the peptide comprises any one of the amino acid residues Ala, Ile, Leu, Phe, Asn, Gln, Pro, Gly or Val.

7. A composition according to claim 1 wherein the peptide comprises any one of the amino acid residues of Ala, Leu, Gln, Pro and Gly.

8. A composition according to claim 1 which comprises the amino acid sequence Leu-Ala-Leu-Leu-Ala (SEQ ID NO: 1) or Leu-Leu-Leu-Ala-Ala-Gly (SEQ ID NO: 6).

9. A composition according to claim 1 which is linked to a monomer, macromer or prepolymer suitable for use in the manufacture of a contact lens.

10. A composition of according to claim 9, wherein the composition comprises a polymerizable vinylic group.

11. A contact lens or ocular implant that comes into contact with tear fluid comprising a composition according to claim 1.

12. A contact lens or ocular implant that comes into contact with tear fluid comprising a preformed contact lens and:
a composition according to claim 1 coated thereon; or
a composition according to claim 9 or 10.

13. A contact lens according to claim 11, wherein the preformed contact lens is composed of a hydrogel material.

14. A contact lens according to claim 13, wherein the contact lens is a silicone hydrogel contact lens comprising a silicone hydrogel material.

15. A contact lens according to claim 11 further comprising a base coating on the preformed contact lens but beneath the composition.

16. A contact lens according to claim 15, wherein the composition is covalently attached to the base coating.

17. A contact lens according to claim 15, wherein the base coating comprises a polymeric coating material.

18. A contact lens according to claim 12 further comprising a plasma coating on the preformed contact lens but beneath the composition.

19. A contact lens according to claim 11 which is a disposable contact lens.

20. A method for the manufacture of a composition which method comprises linking a peptide to a lubricant, wherein the peptide is cleavable by one or more proteinases present in tear fluid and, optionally, linking the resulting peptide-lubricant conjugate to a monomer, macromer or prepolymer suitable for use in the manufacture of a contact lens.

21. A method for the manufacture of a composition which method comprises linking a peptide to a monomer, macromer or prepolymer suitable for use in the manufacture of a contact lens, wherein the peptide is cleavable by one or more proteinases present in tear fluid and linking the resulting peptide-monomer, -macromer or -prepolymer conjugate to a lubricant.

22. A method according to claim 20 or 21, wherein the lubricant is hyaluronic acid, a cellulose derivative, a dextran, a polymeric alcohol, a polyvinyl alcohol or povidone (polyvinylpyrrolidone)

23. A method for the manufacture of a contact lens which method comprises:
providing a preformed contact lens; and
coating said contact lens with a composition according to claim 1.

24. A method for the manufacture of a contact lens which method comprises:
preparing a preformed contact lens in the presence of a composition according to claim 9.

25. A method for the manufacture of a contact lens which method comprises:
preparing a composition according to claim 20 or 21 and preparing a preformed lens in the presence of the resulting composition.

26. A composition comprising a monomer, macromer or prepolymer suitable for use in the manufacture of a contact lens and a peptide cleavable by one or more proteinases present in tear fluid.

27. A composition according to claim 26, wherein the monomer, macromer or prepolymer comprises a polymerizable vinylic group.

28. A contact lens or ocular implant that comes into contact with tear fluid comprising a composition according to claim 26.

29. A contact lens according to claim 28, wherein a lubricant is linked to the contact lens through the peptide.

30. A method for the manufacture of a contact lens which method comprises:
preparing a preformed contact lens in the presence of a composition according to claim 26 and;
linking a lubricant to the thus formed preformed contact lens via the peptide.
